(19) United States
(12) Patent Application Publication
(10) Pub. No.: US 2012/0157735 A1
(43) Pub. Date: Jun. 21, 2012

(54) SUPPORTED MESOPOROUS AND MICROPOROUS MATERIAL, AND PROCESS FOR PRODUCING THE SAME

(76) Inventors: Xinhe Bao, Dalian (CN); Ding Ma, Dalian (CN); Wenjie Shen, Dalian (CN); Lijun Gu, Dalian (CN)

(21) Appl. No.: 13/380,323

(22) PCT Filed: Jun. 23, 2009
(86) PCT No.: PCT/CN09/00694

§ 371 Date: Mar. 8, 2012

(51) Int. Cl. C07C 2/76; B01J 29/40; B01J 29/076; B01J 29/78; B01J 29/48; B01J 37/02; B01J 37/08; B01J 29/06; B01J 29/70; B82Y 99/00

(52) U.S. Cl. 585/415; 502/60; 502/77; 502/64; 977/902

(57) ABSTRACT

A process for producing a supported mesoporous and microporous material, comprises contacting a support with a template to produce a supported template, and contacting the supported template with one or more microporous material precursor to produce a supported microporous material-template composite, and subsequently removing the template from the supported microporous material-template composite to produce the supported mesoprous and microporous material. A composition comprising a supported mesoprous and microporous material produced by this process can be used for methane dehydroaromatization.

This invention relates to porous materials and their production, and more specifically to porous materials having pore widths or diameters of the order of ten to one hundred nanometers.

Catalysts that are routinely employed in industrial petrochemical and refining processes need to be properly shaped to meet the mechanical and mass transfer requirements of the process. Often, this is achieved by mixing active catalyst components with oxide binders such as α-alumina, silica, alumina-silica or zirconia, so as to improve strength and attrition resistance. However, the binders can often cause blocking of the active catalyst components. They can also act to hinder diffusion of reactant and product molecules to and from the active catalyst components. Additionally, in hydrocarbon conversion processes, acidic binders can cause the deposition of carbonaceous deposits, particularly at high temperatures.

One way in which porosity can be introduced to a material is exemplified by the catalyst USY used in fluidised catalytic cracking of heavy crude oil refinery fractions, in which a zeolite catalyst (zeolite Y) is mixed with an alumina binder, and steamed at high temperature to introduce porosity to the alumina component, and also partially to disrupt the zeolite framework in order to make the active sites within zeolite micropores more available to the reactants.

US 2005/0152834 describes the preparation of a zeolite with mesoporosity by adding a carbon aerogel, comprising carbon spheres, into a zeolite synthesis gel, and by subsequently removing the carbon by combustion.

In Chem. Rev., 2006, 106, pp 896-910, and Chem. Mater., 2001, 13, pp 4416-4418, the preparation of zeolites with mesoporosity using carbon nanotubes is described.

However, there remains a need for alternative materials and processes for preparing mesoporous materials, in particular supported catalysts with mesoporosity.

According to the present invention, there is provided a process for producing a supported mesoporous material, which process comprises contacting a support with a template to produce a supported template, and contacting the supported template with one or more microporous material precursor to produce a supported microporous material-template composite, and subsequently removing the template from the supported microporous material-template composite to produce the supported mesoporous and microporous material.

In the present invention, mesoporosity can be introduced to a material by forming or depositing the material around a template. The template dimensions are such that, when it is removed, the microporous material remaining after the template removal comprises mesopores. Mesopores are typically defined as pores with a width or diameter of 2 nm or more, and typically up to 100 nm. Preferred templates are organic compounds or carbon, which can be completely removed from the material through processes such as combustion, or calcination in an oxygen-containing atmosphere. In a typical procedure, removal of the template is achieved through calcination in an oxygen-containing atmosphere at a temperature above 400° C., preferably 500° C. or above to ensure efficient removal of the template. Calcination is usually carried out at temperatures of 800° C. or below, for example 650° C. or below, as higher temperatures can cause decomposition of the mesoporous material.

The pores to be produced typically have a size in the range of from 2 to 100 nm in at least one dimension, for example a width or diameter of 3 to 100 nm. The template therefore typically has corresponding dimensions. In a preferred embodiment of the invention, the template is rod-like or elongated in one dimension, which creates tubular pores within the material on its removal, the width or diameter of the rods/tubes being in the range of from 2 to 100 nm. This type of pore is preferred over shapes such as spheres, for example, as it reduces the extent of template that is inaccessible to the surface of the material once formed around the template. With spherical templates, for example, some of the spheres may be located on the surface of the templated material, while other spheres may be not be accessible to the surface, and hence will not be removed during the template removal process. With rod-like templates, there is continuity to the surface, and hence removal is more efficient, as is the extent of porosity that can be introduced into the material. In a preferred embodiment of the present invention, the template is carbon nanotubes.

Introducing mesoporosity into a material is advantageous in applications such as catalysis. For example, non-porous materials suffer from low active surface areas, which limit the amount of active catalyst that is available for contact with reactants at any one time. Microporous materials, for example zeolites, have comparatively higher surface areas, but the relatively small dimensions of the microporous channels and pores can limit the quantity and size of reactant and reactant molecules that can diffuse into and out of the microporous structure. The introduction of mesoporosity can improve the quantity of catalytically active sites available on the external surface of a material per unit area or volume of that material, and additionally can improve the rate of diffusion of reactant molecules to, and product molecules from, the catalytically active surfaces.

The mesoporous material is typically an oxide or other ceramic material that is resistant to the conditions of the template removal, for example through combustion. Examples include amorphous and crystalline oxides comprising one or more of Si, Al, Zr, Hf, Ce, P, Mg, V, Zn, Mn, Ga, Nb, Ta, Fe, Sn, Li, Be, B, Ge, As, Co, Zn and Ti.

In one embodiment, the mesoporous material is or comprises a zeolite, for example one or more zeolites comprising one or more of the elements Si, Al, Ga, Ge, B, Ti, Co and P incorporated into the zeolite framework, and optionally additionally comprising further elements not incorporated into the zeolite framework, but instead being present within the zeolite micropores, for example metals such as transition metals, rare earth metals, alkali metals, alkaline earth metals and main group metals, optionally in an oxide form. Thus, the mesoporous material can have additional porosity for example microporosity as exhibited by zeolites, in which the pores having a width or diameter typically 2 nm or less, for example 0.3 to 1.5 nm. Non-framework elements can be present in the material before being contacted with the template, or they can be introduced at the same time as the material or precursors thereof are contacted with the template, or can be introduced subsequently after the template has been removed.

The mesoporous material is produced from one or more precursor compounds. The one or more precursors are contacted with the template and treated, typically at elevated temperatures, such that a solid forms or deposits around the template to produce a mesoporous material-template composite, which leaves the mesoporous material when the template is removed. In one embodiment, one or more of the precursor compounds can be small particles of the desired mesoporous material composition, which deposit on the surface of the template and cross link during treatment such as thermal treatment to form an extended cross-linked material around the template as the mesoporous material-template composite. This technique can be used where the desired mesoporous material is a ceramic material such as an oxide or carbide, and small particles of the non-mesoporous oxide or carbide are available. The small particles as precursors are typically thermally treated at high temperatures to form a cross-linked network around the template. Temperatures typically in excess of 550° C., for example 600° C. or more or 700° C. or more are used, and typically up to 1500° C., for example up to 1300° C. or up to 1100° C. Such heating is typically carried out in under non-oxidative conditions, for example in the absence of oxygen. Templates stable to such conditions, include carbon templates, such as carbon nanotubes. In one embodiment in which such a precursor is used, the mesoporous material is silicon carbide, and the small particles are silicon carbide particles with particle widths or diameters significantly smaller than the desired size of the mesopores.

In another embodiment, the one or more precursor compounds of the mesoporous material are chemically different from the desired mesoporous material, and instead are compounds that comprise the desired elements that are to be present in the mesoporous material. For example, where the desired mesoporous material is an aluminosilicate zeolite, the template is contacted with the zeolite synthesis gel, which typically comprises an aqueous mixture of a silicon source (for example alkoxy silanes, silicate salts, for example sodium silicate, or colloidal silica), a source of aluminium (for example an aluminium halide, an alkoxy aluminium compound, or an aluminate salt such as sodium aluminate), a base such as sodium or potassium hydroxide, and an organic amine or organic ammonium salt. The conditions are then maintained such that the zeolite desposits on the template, which forms a zeolite having mesoporous characteristics once the template is removed. For zeolite synthesis and synthesis of other oxide materials for example, hydrothermal conditions are often used, wherein the one or more precursors and a template are heated in a sealed bottle, vessel or autoclave at temperatures typically in excess of 100° C., for example in the range of from 100 to 300° C. Templates stable to such conditions include carbon, for example carbon nanotubes.

In one embodiment, the mesoporous material is a catalyst active for methane dehydroaromatisation to one or more aromatic compounds, for example selected from one or more of benzene, toluene and xylene.

Methane dehydroaromatisation catalysts can comprise one or more zeolites modified with one or more metals active for methane dehydroaromatisation, for example one or more of Mo, Re and W, preferably Mo, which are preferably present in the zeolite at a loading in the range of from 0.1 to 20 wt %, for example in the range of from 1 to 10 wt %. Optionally, the methane dehydroaromatisation catalyst metals can be associated with one or more further or additional metals. Where Mo is used as a methane dehydroaromatisation catalyst metal, preferred additional metals include one or more of Ru, Pt, W, Ze, Co, Fe and Cr. Where W is a methane dehydroaromatisation catalyst metal, Zn is a preferred additional metal. Where present, additional metals are typically present in the zeolite at a loading in the range of from 0.1 to 20 wt %, for example in the range of from 0.1 to 10 wt %.

Zeolites that can be used in methane dehydroaromatisation catalysts, and which can be the mesporous materials of the present invention, include those having pores in one or more dimensions with rings formed of at least 10 non-oxygen framework atoms. For brevity, such a pore size will henceforth be referred to as a 10-membered ring. Pore structures with smaller than 10-membered rings are believed to be too small to allow passage of aromatic compounds. A database of zeolite structures is maintained by the International Zeolite Association.

Examples of suitable zeolite structures include MFI and MWW structures, which both have 10-membered ring pores. In the case of MFI, the pore structure is three dimensional. There are two channel systems having 10-membered ring sizes. One of the 10-membered ring channels or pores is linear, the other is sinusoidal. In the case of the MWW structure, the pore structure is two-dimensional, the pores in each dimension being formed of 10-membered rings. The pores intersect at cages which are formed from 12-membered rings.

Zeolites active for methane dehydroaromatisation generally have Brønsted acid characteristics. Brønsted acidity in a zeolite arises when the overall charge on the zeolite framework is negative, and protons or H₃O⁺ ions act as the counter-cations to the framework negative charge. Such framework negative charge is found in aluminosilicate and silicoaluminophosphate zeolites, for example. The zeolite framework can additionally comprise other elements, such as boron, cobalt, titanium, gallium or germanium. Aluminosilicate zeolites tend to exhibit stronger acidity compared to silicoaluminophosphates, for example. Stronger Brønsted acidity is advantageous for methane dehydroaromatisation, as methane conversions are typically higher. In a preferred embodiment of the invention, the zeolite is an aluminosilicate zeolite adopting the MWW of MFI structure, for example MCM-22, MCM-49 or ZSM-5. The silicon/aluminium molar ratio of aluminosilicate zeolites is suitably in the range of from 1 to 150, and is preferably in the range of from 15 to 40.

One or more metals can be incorporated into a zeolite either during synthesis of the zeolite, or by modifying the zeolite after its synthesis, typically through ion exchange or by impregnation. For methane dehydroaromatisation catalysts, it has been found that the most active form of the metal is where it is present as counter-cation to the negative framework charge. Where the metal is present in a condensed form, for example where the catalyst metal is in the form of metal-oxide particles located either within the internal microporous zeolite structure or on the external surface of the zeolite structure, for example within the zeolite mesopores, lower activity results. Therefore, in a preferred embodiment of the invention, the catalyst metal is present, at least in part, in the form of discrete ions within the zeolite microporous structure.

Where the mesoporous material, for example a zeolite, is supported on a support, the mesoporous material typically comprises in the range of from 0.5 to 40% by weight of the total composition.

A typical zeolite synthesis mixture comprises sources of the framework atoms, and a so-called structure-directing agent, usually an organo-amine compound. The structure directing compound is provided in one embodiment in the form of a hydroxide, for example a quarternary ammonium hydroxide having one to four organic groups on the nitrogen atom. Additionally, or alternatively, other hydroxides can be present, for example inorganic hydroxides such as sodium or potassium hydroxide, or other quarternary ammonium hydroxides having one to four organic groups on the nitrogen atom.

The source of zeolite framework atoms can be in the form of small oxide particles, for example in the form of a colloidal suspension, or in the form of one or more water soluble compounds or salts, for example alkoxide compounds, halide salts, oxalate salts, carbonate salts or nitrate salts.

In a typical zeolite synthesis, a mixture comprising a source of silicon, a source of aluminium, and tetrapropylammonium hydroxide, is heating in a sealed vessel to a temperature in the range of from 100 to 300° C., typically in the range of from 150 to 250° C. As a result of the heating, the pressure in the sealed vessel increases to above ambient, the final pressure being dependent on the composition of the zeolite synthesis mixture, on the temperature, and on the air space in the sealed vessel that is not initially occupied by the zeolite synthesis mixture. Typically, the zeolite crystallises over a period of several hours, for example in the range of from 1 to 200 hours. The catalyst is then separated from the zeolite synthesis mixture, typically by filtration, decantation or centrifugation.

The resulting solid is then typically washed with water to remove excess zeolite synthesis mixture. Often, washing is continued until the pH of the wash-water falls below a certain level, for example below pH 8 or until pH 7 is reached. The washed solid can then be dried, typically at above-ambient temperatures such as up to 200° C., to remove residual water. The resulting zeolite can then be calcined in an oxygen-containing atmosphere at temperatures usually in the range of from 450 to 650° C. to burn-off any organic material, for example organoamine components originating from the zeolite synthesis mixture.

One or more additional metals, for example methane dehydroaromatisation catalyst metals and/or optional additional metals, can also be present in the zeolite synthesis mixture, being present in the form of one or more soluble compounds or salts, such as alkoxide compounds, as halide salts, as oxalate salts, as carbonate salts or as nitrate salts.

Alternatively, the one or more catalyst metals and/or optional additional metals can be incorporated into the catalyst after the calcination stage, for example through impregnation or ion exchange techniques. Such procedures can be carried out on the mesoporous zeolite itself, or on a supported mesoporous zeolite.

Ion-exchange of a zeolite can be achieved by suspending the zeolite in a solution of one or more cations, optionally at elevated temperature and/or pressure, followed by filtration and drying. This procedure can then be repeated if necessary until the desired loading of cations (for example methane dehydroaromatisation catalyst metals and/or additional metals) is achieved.

Impregnation can be carried out by suspending the zeolite in a solution of one or more cations, and then evaporating the solution to dryness.

In zeolite synthesis, the synthesis mixture is typically an aqueous mixture comprising sources of the zeolite framework constituent elements. For aluminosilicate zeolites, the synthesis mixture comprises sources of silicon and aluminium. These are either dissolved in the (usually aqueous) solvent or are suspended therein. Silicon is often provided in the form of a tetraalkoxysilane such as tetraethoxysilane, or as a sodium silicate solution or silica or silicate colloid. Examples of suitable aluminium sources include aluminium chloride and sodium aluminate.

The zeolite synthesis mixture typically additionally comprises one or more organoamine salts, often hydroxides, which can act as structure-directing agents for the microporous structure of the zeolite. Additionally, ammonia or an organoamine hydroxide salt is often added to adjust pH. The pH of zeolite synthesis solutions is typically in the range of from 8 to 11, for example from 9 to 10. Additional amines or organoamine salts can also be added as structure directing agents. In ZSM-5 synthesis, for example, one or more tetrapropylammonium salts, typically hydroxide, are added to the synthesis mixture.

In one embodiment of the invention, the mesoporous material can be supported, for example on an oxide support, such as a support selected from one or more of silica, alumina, silica alumina, zirconia, hafnia, magnesia, titania, ceria, zeolites, other ceramic materials such as carbides or nitrides, for example silicon carbide.

The support can be in the form of particles, such as spheres, granules or extrudates. The support can in another embodiment be in the form of a monolith or foam.

Silicon carbide is a hard, attrition resistant compound, and is resistant to coke deposition in a number of hydrocarbon conversion reactions, for example the dehydroaromatisation of methane to one or more aromatic compounds such as benzene, toluene and xylenes. Silicon carbide can also act as a strong support for oxide materials, for example amorphous and crystalline forms of oxides comprising one or more of silicon, aluminium, phosphorus, This is because it can be pre-treated in oxygen to form Si—O species on the SiC surface, which can form strong bonds with the oxide materials to be supported. Typically, the pre-treatment of the silicon carbide to form the surface oxygen species is carried out at a temperature in the range of from 600 to 950° C.

Using SiC as a support for methane dehydroaromatisation catalysts can help achieve higher methane conversions per mole of methane dehydroaromatisation catalyst metal compared to the SiC-free catalysts. In the case of ZSM-5-containing catalysts, improved catalyst lifetime can result when the catalyst comprises silicon carbide, compared to corresponding catalysts which do not comprise silicon carbide, as described for example in PCT patent application PCT/CN2008/000978.

To make the supported mesoporous material, the mesoporous material-template composite or the mesoporous material after template removal can be added to the support to make the supported mesoporous material. Alternatively, the support, the precursors, and the template can be mixed simultaneously. Alternatively the template can first be added to or supported on the support and the supported template contacted with the mesoporous material precursors.

Chemical Vapour Deposition (CVD) methods can be used to deposit a template on a support. For example, carbon nanotubes can be deposited on a support, for example silicon carbide, by decomposition of ferrocene.

In methane dehydroaromatisation, a feedstock comprising methane is contacted with the catalyst under conditions of elevated temperature and optionally elevated pressure. The reaction temperature is suitably in the range of from 400 to 900° C., and is typically in the range of from 600 to 850° C. The pressure is typically in the range of from 1 to 80 atm, for example in the range of from 1 to 50 atm, such as in the range of from 1 to 25 atm. Optionally, methane is not the sole component of the feedstock, and in one embodiment an inert diluent such as nitrogen or argon is additionally present. In a further embodiment, where a diluent is present, the methane concentration in the feedstock is in the range of from 80 to 99.9% by volume.

The catalyst is typically in the form of a fixed bed, with the methane-containing feedstock being passed over the catalyst. Typically, the GHSV (Gas Hourly Space Velocity, in units of mL gaseous feedstock corrected to standard temperature and pressure, per g catalyst, per hour) of the total feedstock is in the range of from 100 to 20 000 mL g⁻¹ h⁻¹, for example in the range of from 100 to 10 000 mL g⁻¹h⁻¹, and more preferably in the range of from 1 000 to 5 000 mL g⁻¹h⁻¹, such as 1000 to 2000 mL g⁻¹h⁻¹.

The products of the reaction are one or more aromatic compounds and hydrogen. The aromatic compounds that can be produced in the reaction include benzene, toluene, one or more xylene isomers (often referred to collectively as “BTX”). By-products include double-ring aromatic compounds such as naphthalene and aliphatic hydrocarbons. Additionally, carbonaceous deposits can also be produced which can cause or contribute to catalyst fouling or coking.

There now follow non-limiting examples illustrating the invention, with reference to the Figures, in which;

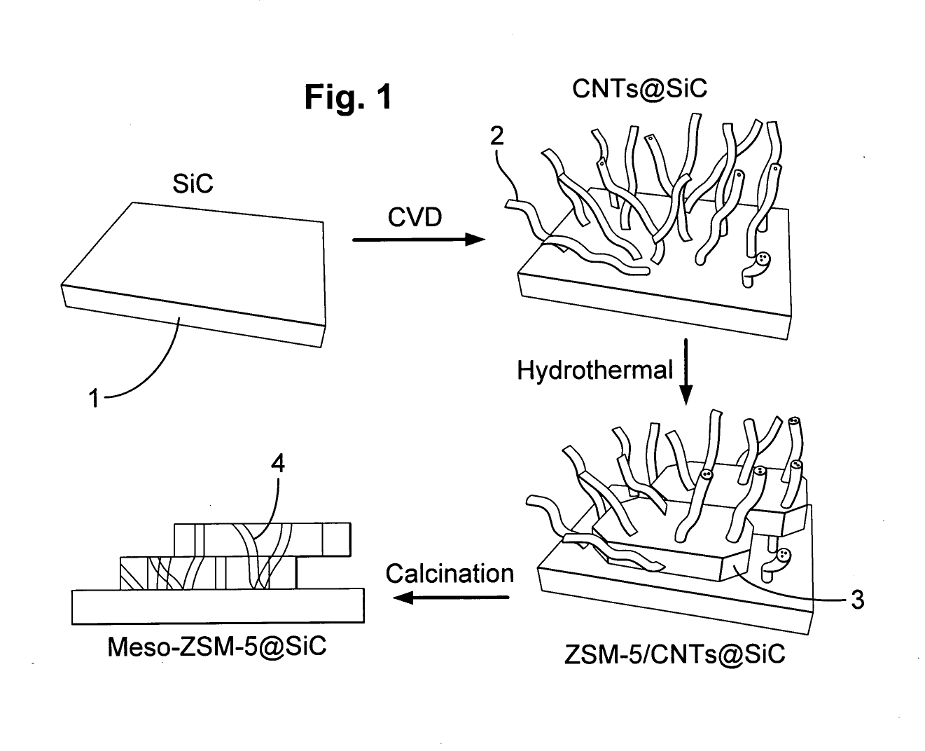

FIG. 1 illustrates the steps to produce a SiC-supported mesoporous Mo-ZSM-5 catalyst.

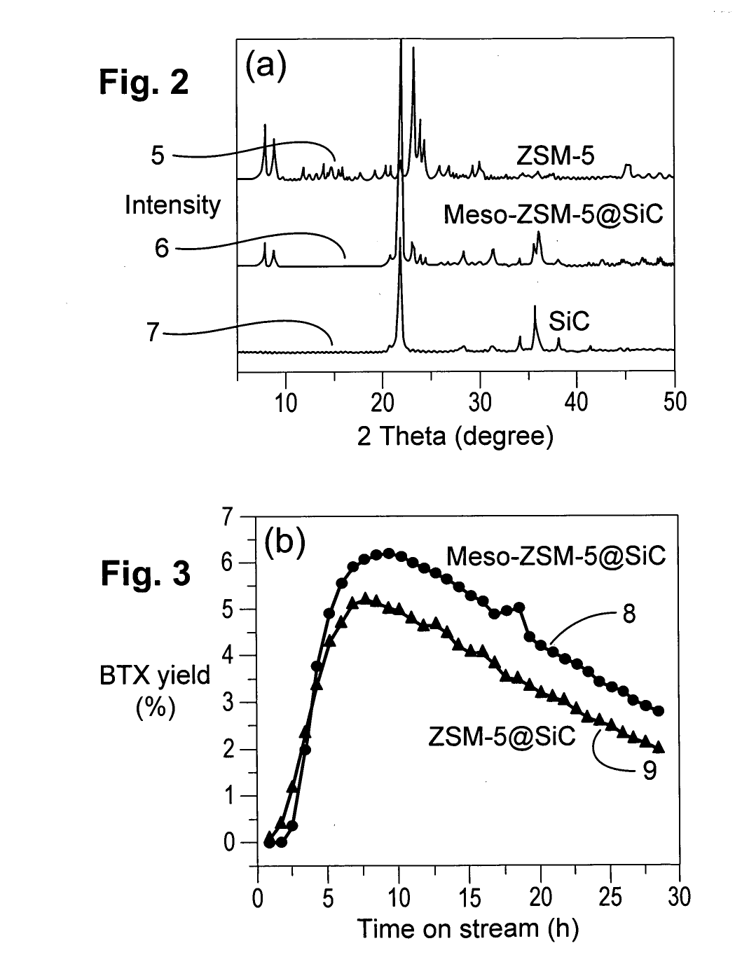

FIG. 2 shows X-Ray Diffractograms of ZSM-5 and mesporous ZSM-5 supported on SiC.

FIG. 3 shows catalytic activity of SiC-supported Mo-ZSM-5 with and without mesopores towards methane dehydroaromatisation.

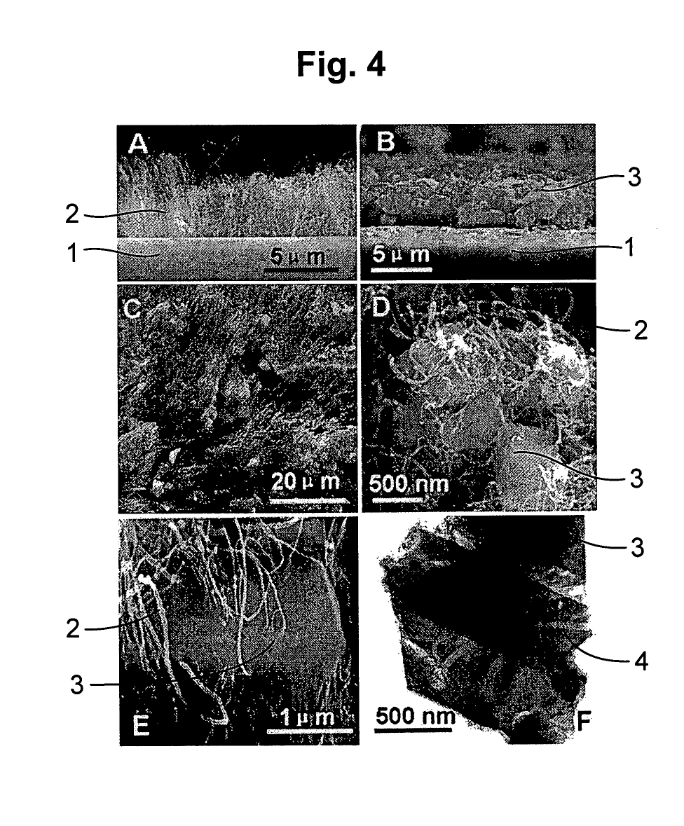

FIG. 4 shows photomicrographs of;

(A) and (C) carbon nanotubes supported on a silicon carbide surface

(B), (D) and (E) ZSM-5 with carbon nanotube template on a silicon carbide surface

(F) Increased magnification of the zeolite crystals after carbon nanotube removal.

FIG. 1 shows schematically a process for preparing a mesoporous supported zeolite, in which carbon nanotubes, 2, are desposited onto a SiC surface, 1, by chemical vapour deposition to form supported carbon nanotubes. Zeolite ZSM-5 crystals, 3, are then formed on the nanotube-modified surface by adding the supported nanotubes to a zeolite synthesis gel and hydrothermally treating the mixture. After calcination, the carbon nanotubes are removed to leave SiC-supported ZSM-5 with mesoporous channels, 4.

FIG. 2 shows XRD (X-Ray Diffraction) plots of; ZSM-5 (5), silicon carbide-supported mesoporous ZSM-5 after carbon nanotube removal (6), and silicon carbide (7). The plots demonstrate that the overall framework crystalline structure of ZSM-5 remains intact, even though mesoporosity has been introduced.

FIG. 3 shows the combined yield of benzene, toluene and xylenes (BTX) from methane with time in the presence of a 6 wt % Mo/SiC-suported mesoporous ZSM-5 catalyst, 8, and a SiC-supported 6 wt % Mo-ZSM-5 catalyst, 9, that was synthesised without the use of carbon nanotubes, resulting in the ZSM-5 being without mesopores. This catalyst was prepared in the same way as the corresponding mesoporous SiC supported Mo-ZSM-5, but without the addition or use of carbon nanotubes in the synthesis procedure. The results show a clear improvement in aromatic yields using the mesoporous-modified catalyst.

FIG. 4 is a series of photomicrographs of various materials associated with the synthesis of SiC-supported mesoporous ZSM-5. FIGS. 4A and 4C show carbon nanotube fibres, 2, supported on a silicon carbide surface, 1. FIGS. 4B, 4D and 4E shows ZSM-5 crystals, 3, on the SiC surface, 1, in which the carbon nanotubes, 2, have not yet been removed. FIG. 4F is a view of ZSM-5 crystals, 3, after removal of the carbon nanotube template, showing the mesoporous channels, 4.

A procedure for preparing the SiC particles or a SiC-wafer was first calcined at 1173 K for 4 h before being allowed to cool. They were then heated from room temperature to 973 K over a period of 70 minutes under nitrogen, with a nitrogen flow rate of 100 cm³/min (volume based at standard temperature and pressure). The nitrogen flow was replaced with a mixture of 10% hydrogen in nitrogen at a flow rate of 400 cm³/min (at standard temperature and pressure). Additionally, a solution of 0.02 g mL⁻¹ ferrocene in xylene was fed at a rate of 0.1 mL min⁻¹ (liquid volume). After 40 minutes' reaction (in the case of Si wafer, the reaction lasts 2 mins), the temperature of the furnace was dropped to room temperature under nitrogen at a flow rate of 100 cm³/min (at standard temperature and pressure). The resulting solid is denoted herein as CNT@SiC.

ZSM-5 was deposited on the CNT@SiC by adding 7 g of CNT@SiC to a zeolite synthesis gel of comprising 0.60 g NaCl, 54 mL H₂O, 6.59 g tetraopropylammonium hydroxide (TPAOH), 3.78 mL tetraethoxysilane (TEOS) and 0.06 g NaAlO₂ (giving a Si/Al molar ratio in the synthesis gel of 25), and stirring the mixture at 30° C. for 4 h. The mixture was placed into a PTFE-lined autoclave, and held at a temperature of 443 K for 2 days. The resulting solid, denoted herein as ZSM-5/CNT@SiC, was separated by filtration, washed with deionized water and dried at 393 K. To remove the carbon nanotubes, calcination was carried out at 823 K for 360 followed by calcination at 973 K for 360 min, to give the final supported mesoporous zeolite, denoted meso-ZSM-5@SiC.

On its XRD pattern (FIG. 2a), the intense peak centered at ca. 21.8° of meso-ZSM-5@SiC and SiC was due to silica formed on the surface of SiC during the calcination at 1173 K before the synthesis.

Metal ion contaminants, produced during nanotube synthesis, and which were present inside the meso-ZSM-5@SiC, were removed by refluxing in concentrated HNO₃ (68 wt %) at 393 K for 12 h twice. The material was then ion-exchanged with 2 mol L⁻¹ NH₄NO₃ solution for 4 h at 353 K twice, and subsequently calcined at 813 K for 5 h to produce the protonated form of ZSM-5. The quantity of ZSM-5 suported on the SiC was calculated by weight increase after hydrothermal synthesis. Molybdenum at a loading in the zeolite of 6 wt % was prepared by impregnating the mesoporous supported zeolite with an aqueous solution containing the desirable amount of ammonia heptamolybdate ((NH₄)₆[Mo₇O₂₄].4H₂O), before drying and subsequent calcination at 773 K for 6 h.

To evaluate catalyst performance, methane dehydroaromatization was carried out in a continuous flow reactor system equipped with a quartz tube (10 mm id) packed with samples which comprised 100 mg of the zeolite. A feed gas mixture of 90% CH₄ with 10% N₂ was purified and then introduced into the reactor at a flow rate of 1500 ml g⁻¹h⁻¹. The reaction was conducted at 1023 K at a pressure of 1 atm. The products were analyzed by an on-line gas chromatograph (Varian CP-3800) equipped with a flame ionization detector (FID) for the analysis of CH₄, C₆H₆, C₇H₈, and C₁₀H₁₂ and a thermal conductivity detector (TCD) for the analysis of H₂, N₂, CH₄, CO, C₂H₄ and C₂H₆. N₂ (10%) in the feed was used as an internal standard for analyzing all products, including carbonaceous deposition on the basis of converted methane molecules.

The high temperature treatments described below were carried out using an electric furnace fitted with a quartz tube.

1-16. (canceled)

17. A process for producing a supported mesoporous and microporous material, which process comprises contacting a support with a template to produce a supported template, and contacting the supported template with one or more microporous material precursor to produce a supported microporous material-template composite, and subsequently removing the template from the supported microporous material-template composite to produce the supported mesoporous and microporous material.

18. A process as claimed in claim 17, in which the template has a size in the range of from 2 to 500 nm in at least one dimension.

19. A process as claimed in claim 17, in which the template is carbon.

20. A process as claimed in claim 19, in which the template is carbon nanotubes.

21. A process as claimed in claim 17, in which the material is a zeolite.

22. A process as claimed in claim 21, in which the material is ZSM-5, MCM-22 or MCM-49.

23. A process as claimed in claim 21, in which the zeolite comprises one or more non-framework elements.

24. A process as claimed in claim 23, in which the zeolite comprises molybdenum as a non-framework element.

25. A process as claimed in claim 17, in which the support is silicon carbide.

26. A process as claimed in claim 17, in which the contacting of the material with the template is carried out under hydrothermal conditions.

27. A process as claimed in claim 17, in which the template is removed by heating in an atmosphere comprising molecular oxygen at a temperature in the range of from 400 to 700° C.

28. A composition comprising a supported mesoporous material, which supported mesoporous material is produced by a process in accordance with claim 17.

29. A composition as claimed in claim 28, in which the mesoporous and microporous material is zeolite ZSM-5, MCM-49 or MCM-22, additionally comprising molybdenum as a non-framework element.

30. A process for converting methane into one or more aromatic compounds, which process comprises contacting methane with a composition as claimed in claim 28.

31. A process as claimed in claim 30, in which the composition comprises silicon carbide as a support.

32. A process as claimed in claim 30, in which the methane is contacted with the composition at a temperature in the range of from 400-900° C. and a pressure of from 1 to 50 atm.
